(19) United States
(12) Patent Application Publication
(10) Pub. No.: US 2009/0311743 A1
(43) Pub. Date: Dec. 17, 2009

(54) Process for the Production, in Prokaryotes, of Active, Stable Transposases of Mariner Mobile Genetic Elements

(75) Inventors: Corinne Auge-Gouillou, Veretz (FR); Yves Bigot, Saint Avertin (FR); Benjamin Brillet, Orvault (FR); Stéphanie Germon, Nouzilly (FR)

Correspondence Address: FOLEY AND LARDNER LLP;SUITE 500, 3000 K STREET NW, WASHINGTON, DC, 20007, US

(73) Assignee: CENTRE NATIONAL DE LA RECHERCHE SCIENTIFIQUE(CNRS)

(21) Appl. No.: 12/085,435

(22) PCT Filed: Nov. 23, 2006
(86) PCT No.: PCT/EP2006/068853

§ 371 Date: May 23, 2008

(30) Foreign Application Priority Data
Nov. 30, 2005 (FR) 0512180

(51) Int. Cl. C12P 21/00; C12N 9/12; C12N 1/00; C12N 1/21; C12N 15/63; C07H 21/00

(52) U.S. Cl. 435/69.1; 435/194; 435/243; 435/252.3; 435/252.33; 435/320.1; 536/25.3

(57) ABSTRACT

The present invention relates to a process for the production, by a prokaryotic host cell, of an active, stable transposase of a Mariner mobile genetic element belonging to the mauritiana subfamily. The invention also relates to an active, stable transposase that can be produced using such a process, and to the molecular biology tools, such as the prokaryotic host cells, the expression vectors or the kits, which make it possible to produce an active, stable transposase, or which use it. In addition, the present invention relates to the uses of the active, stable transposases.

This invention relates to the field of molecular biology dealing with transposable elements. More particularly, the invention relates to improving the properties of natural transposases of mariner mobile genetic elements for their use in biotechnology.

The invention also relates to a process for the production, by a prokaryotic host cell, of active, stable transposases of a mariner mobile genetic element belonging to the mauritiana sub class.

The invention further relates to an active, stable transposase that can be produced by means of such a process, as well as the molecular biology tools, such as prokaryotic host cells, expression vectors and kits, needed for the production and application of an active, stable transposase.

Moreover, this invention covers the uses of active, stable transposases.

Transposable elements (TE) or mobile genetic elements (MGE) are small-sized DNA fragments capable of moving from one chromosome site to another (Renault et al., 1997). These DNA fragments are characterised by Inverted Terminal Repeats (ITRs) located in the terminal 5′ and 3′ positions. An enzyme coded for by the TEs themselves, transposase, catalyses the transposition process of the latter.

TEs have been identified both in prokaryotes and eukaryotes (refer to a reference book in the field: Craig et al., 2002).

TEs are divided into two categories according to their transposition mechanism. On the one hand, category I elements, or retrotransposons, transpose via reverse transcription of an RNA intermediate. On the other hand, category II elements transpose directly from one chromosome site to another via a DNA intermediate according to a “cut-paste” mechanism.

A large number of TEs have been identified to date in prokaryotes, for example insertion sequences such as IS1, and transposons, such as Tn5.

In eukaryotes, category II elements consist of 5 families: P, PiggyBac, hAT, helitron and Tc1-mariner.

Mariner mobile genetic elements (or MLE for mariner-like elements) make up a large group of category II TEs belonging to the Tc1-mariner super family (Plasterk et al., 1999)

The ability of TE transposases to mobilise DNA fragments of varying length, homologues or heterologues, containing the sequences of interest for insertion into target nucleic acids, particularly in the host chromosome, has been and continues to be widely used in the field of biotechnology, mainly in the field of genetic engineering.

Amongst the TEs, MLEs have particularly advantageous properties for use in biotechnology, namely in genetic engineering and functional genome engineering. For example, the following properties can be cited in a non-limiting manner:

i) MLEs are small sized transposons that are easy to handle.

ii) The mechanism of MLE transposition is simple. In fact, the transposase itself is capable of catalysing all the steps in the MLE transposition. It is moreover necessary and sufficient to ensure the mobility of MLEs in the absence of host factors (Lampe et al. 1996)

iii) MLEs are characterised by being very widespread amongst prokaryotes and eukaryotes. The first MLE, Dmmuar1, also called Mos1, was discovered in Drosophila mauritiana by Jacobson and Hartl (1985). Following this, many related elements were identified in the genomes, particularly in bacteria, protozoans, fungi, plants, invertebrates, cold blooded vertebrates and mammals.

iv) The transpositional activity of MLEs is highly specific and does not give rise to “resistance” mechanisms of the host genome, such as interference phenomena by methylation [MIP; Jeong et al. (2002); Martienssen and Colot (2001)] or via RNA [RNAi; Ketting et al. (1999); Tabara et al. (1999)]. Transposition events can be controlled by various factors such as temperature, the presence of certain divalent cations and pH.

Consequently, the potential applications of MLEs in biotechnology, mainly as genetic recombination tools, are considerable. Typically, for in vitro insertional mutagenesis applications, the gene coding for the transposase is replaced by a “label” DNA. The transposase has to be supplied as the trans type in protein form. For in vivo or in vitro gene transfer applications, the gene coding for the transposase is replaced by the exogenous DNA to be transferred. The transposase must be supplied as the trans form via an expression plasmid, messenger RNA or the protein itself.

It is therefore essential, in each of these applications, to have available an active transposase in sufficient quantities. The transposase is actually necessary and sufficient for the totality of the transposition process. Nevertheless, in addition to its ability to mediate transposition, MLE transposase is also capable of auto-proteolysis. This irreversible process is likely, by means of a self-regulation mechanism by the transposase itself, to play a role in deciding the maximum amount of active transposase present in a cell. Thus it would seem that the OPI phenomenon (over-production inhibition) described by Lohe et al. (1996), according to which the frequency of Mos1 transposition decreases when the transposase is over-expressed in a cell, is the result of auto-proteolysis of the over-expressed transposase.

This is why MLE transposases pose a number of difficulties in practise in terms of quality and stability. This is particularly true for applications such as in vitro transposition or when the transposase itself (i.e. in protein form) is supplied to cells for ex vivo transposition.

Thus throughout the world, both in industry and in research laboratories, teams which use eukaryotic transposases (mariner or other) are confronted with a recurrent problem: transposases are unstable when they are produced in a prokaryotic system (typically a bacterial system). Batches of purified transposases are generally contaminated by proteolytic fragments of the protein. These fragments are specific to each transposase. Although this problem of transposase instability is not reported in the scientific literature, it has up until the present severely restricted the practical benefits of natural MLEs insofar as both industry and researchers have to have at their disposal effective and stable transposases in order to reduce the number of manipulations, cost and time needed to carry out the required transpositions.

This explains why production in a prokaryotic system, especially in bacteria which remains the most straightforward and least costly way of obtaining the required amounts of transposase, is still not exploited very much, especially in industry.

It is therefore of primary importance to increase the stability of transposases produced in a prokaryotic system.

It is precisely to this need that the present invention addresses itself by supplying means to stabilise MLE transposases produced in a prokaryotic system and, more especially, in bacteria.

Thus a first aspect of the present invention relates to a process for the production by a prokaryotic host cell of an active, stable transposase of a mariner mobile genetic element belonging to the Mauritiana subfamily, comprising at least the following steps:

a) cloning of the nucleotide sequence coding for said active transposase in an expression vector,

b) cloning of a nucleotide sequence coding for the active catalytic sub-unit of cAMP-dependent protein kinase (pKa) in an expression vector,

c) transformation of said host cell with said expression vectors,

d) expression of said nucleotide sequences by said host cell, and

e) obtaining the active and stabilised transposase by pKa phosphorylation.

The term “prokaryotic host cell” here is defined in accordance with the accepted meaning in the field. Preferably, it relates to a bacterial cell. For example, the man skilled in the art can advantageously choose Escherichia coli cells.

The terms and expressions “activity”, “function”, “biological activity” and “biological function” are equivalent and correspond to the accepted meaning in the technical field of the invention. In the precise context of the invention, the activity in question is the enzyme activity of a transposase (“transposase activity” or “transposase function”).

The transposases of interest within the scope of this invention are “active transposases”, in other words transposases capable of mediating the transposition of an MLE. Advantageously, they can be hyperactive if transposition activity has been improved by directed mutagenesis. In this case, the term used is “hyperactive mutant transposases”. Such transposases were described in French patent application no. FR 2 850 395 (filed 28 Jan. 2003).

The term “stable transposase” refers to a transposase whose auto-proteolysis is significantly reduced, and advantageously prevented. More generally, this refers to “inhibition”. Preferably, a “stable” transposase is such that at least 70%, preferably at least 75%, even more preferably at least 80%, at least 85%, at least 90% and most preferably between 95% and even 98% or above (ideally 100%) of proteolysis at each site is prevented. Auto-proteolysis (also called “proteolysis” here) of the transposases involves the active site, in other words a sequence carrying the proteolysis activity (in Mos1 transposase, amino acids 1-116 in FIG. 2) and between 1 and 3 “cleavage sites”, in other words between 1 and 3 target sequences of proteolytic cleaving. In the Mos1 transposase, two main cleaving sites have been identified. They are positioned between amino acids 80/81 and amino acids 101/102. A minor cleavage site is located between amino acids 169/170 (see FIG. 2).

The term “Mauritiana subfamily” includes MLEs whose transposases are coded for by sequences presenting, all along their length or for regions coding for the N- and C-terminal domains uniquely, a sufficient level of homology, in other words at least 75%, in order to be included in the same line as the 4 sequences above in the course of phylogenetic studies carried out using parsimony and neighbour joining methods on a set of data for 1000 sub-samples [See Felsenstein (1993) and Augé-Gouillou et al., (2000)]:
Mos1 transposase (FIG. 2, EMBL access number: X78906)
Mdmar-1 transposase from Mayetiola destructor (EMBL access number: U24436)
Btmar-1 transposase from Rombus terrestris (Bonnin et al., 2005), and
Momar-1 transposase from Metaseuilius occidentalis (EMBL access number: U12279).

Preferably, the mobile genetic element considered here is Mos1.

The term “nucleotide sequence” or “nucleic acid” according to the invention is in accordance with the accepted meaning in the field of biology. These two terms cover DNA and RNA interchangeably, the former for example being genomic, plasmodic, recombinant or complementary (cDNA) and the latter being messenger (mRNA), ribosomal (rRNA) and transfer (tRNA). Preferably, the nucleotide sequences and nucleic acids of the invention are DNA.

The term “nucleotide sequence coding for the active catalytic sub-unit of cAMP-dependent protein kinase (pKa)” was described by Strausberg et al. (2002). In particular it can be obtained from the NBRF-PIR database under access number AAH 54834.

The set of steps implemented in the scope of the process of the invention call on conventional techniques known to the man skilled in the art (see for example Sambrook and Russel, 2001 or Ausubel et al., 1994). In particular, the term “transformation” in this case carries a generic meaning in that it also covers, in addition to transformation in the strict sense, transduction by a viral vector and transfection, molecular biology techniques that are fully known to the man skilled in the art.

According to a particular embodiment, the process of the invention also includes a purification step of the active, stable transposase obtained in step e). Typically, this purification step consists in purification, using common methods known to the man skilled in the art, of the protein fraction with the desired enzyme activity but not necessarily the enzyme itself. The terms “pure enzyme” or “pure transposase” can therefore be used interchangeably to designate the purified active protein fraction or, if relevant, the purified enzyme. At the end of the purification step, the presence of small amounts of contaminating substances, including other proteins, is not necessarily excluded as long as the activity of the transposase in question remains intact and only this activity is detected. Detection of the enzyme activity of interest can be carried out using conventional methods known to the man skilled in the art (Ausubel et al., 1994).

According to another embodiment, cloning in steps a) and b) is carried out in a single expression vector. This can be referred to as “co-cloning” of the nucleotide sequence coding for the active transposase and the nucleotide sequence coding for pKa. In this case, the expression of the two nucleotide sequences is advantageously under the control of the same regulation elements.

A second embodiment of this invention relates to an active, stable transposase of a mariner mobile genetic element belonging to the mauritiana subfamily obtainable by a process such as that described above.

In a third embodiment, this invention is directed to a prokaryotic host cell as defined above which includes at least:

a) the nucleotide sequence coding for the active transposase of a mariner mobile genetic element belonging to the mauritiana subfamily; and

b) the nucleotide sequence coding for pKa.

Advantageously, the active transposase can be a hyperactive mutant transposase, as described above.

In a particular embodiment, nucleotide sequences a) and b) are cloned in expression vectors. Alternatively, these sequences are cloned in a single expression vector.

A fourth aspect of this invention is related to an expression vector wherein it includes at least:

a) the nucleotide sequence coding for an active transposase of a mariner mobile genetic element belonging to the mauritiana subfamily; and

b) the nucleotide sequence coding for pKa.

Here again, the active transposase can advantageously be a hyperactive mutant transposase as described above.

In a fifth embodiment, this invention relates to a kit including at least one active, stable transposase according to the invention.

For example, such a kit can moreover include one or more elements chosen from among mariner Mos1 pseudotransposons (DNA), a buffer compatible with the transposase(s), a stop buffer to stop the transposition reaction, one or more control DNAs (reaction controls), oligonucleotides of use in sequencing after the reaction, viable bacteria, etc.

Other aspects of the invention relate to uses of the tools described above.

Thus, this invention relates to the use of at least one active, stable transposase in accordance with the preceding description for in vitro transposition of a transposable DNA sequence of interest in a target DNA sequence.

This invention also relates to the use of at least one active, stable transposase as defined above for in vivo transposition of a transposable DNA sequence of interest in a target DNA sequence.

The invention further concerns the use of at least one active, stable transposase according to the invention for preparation of a medicament resulting from in vivo transposition of a transposable DNA sequence of interest in a target DNA sequence.

For example, the invention proposes a process for the preparation of a medicament comprising at least one transposition step of a transposable DNA sequence of interest in a target DNA sequence, said transposition being mediated by at least one active, stable transposase according to the invention. The medicament can thus be prepared ex vivo if transposition is carried out in vitro or it can be carried out in situ if the transposition takes place in vivo.

These applications generally involve use of an in vitro or in vivo transposition, methods known to the man skilled in the field of the invention (Ausubel et al., 1994; Craig et al., 2002). More particularly, with regard to in vivo transposition, the target DNA sequence is typically the host genome which can be an organism, eukaryote or prokaryote, or tissue from an organism or even a cell from an organism or tissue.

Another application concerned by this invention relates to the use of a kit according to the invention for insertional mutagenesis and/or sequencing and/or cloning. This involves conventional molecular biology techniques known to the man skilled in the art for which the methods of the invention are found to be of great benefit.

The following figures are given for the purpose of illustration only and in no way limit the scope of this invention.

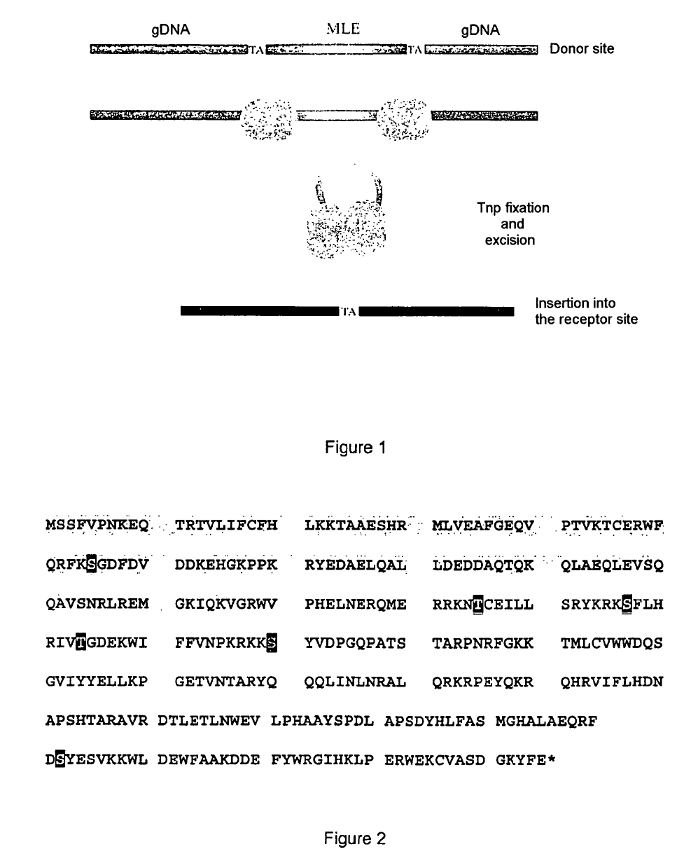

FIG. 1: Diagrammatic representation of MLE transposition. TMP: transposase, gDNA: genomic DNA.

FIG. 2: Protein sequence of Mos1 transposase. Amino acids between which cleaving is carried out (cleavage sites) are highlighted in light grey. Possible phosphorylation sites by pKa are underlined and given in dark grey. The region which potentially carries proteolytic activity (active site) is shaded in white.

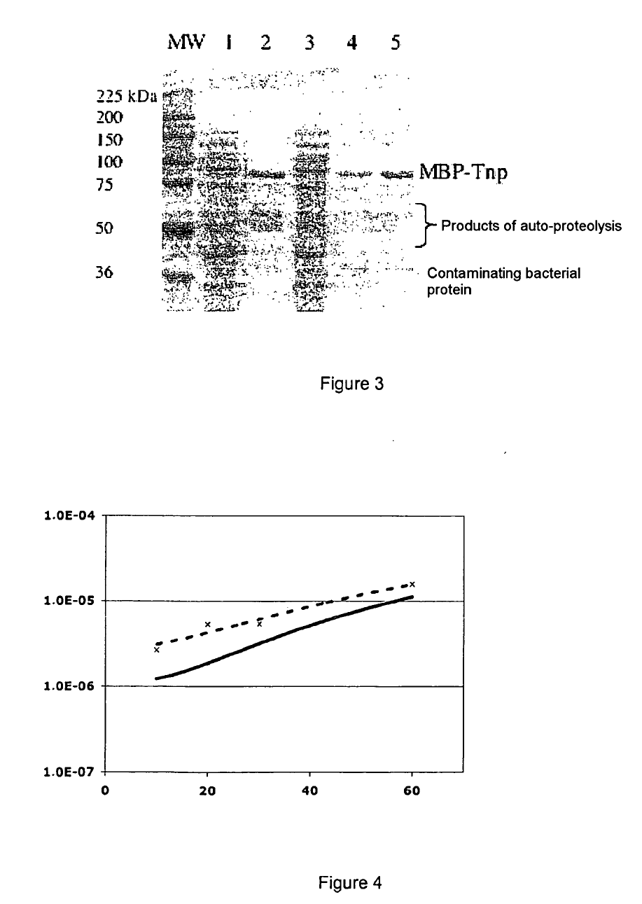

FIG. 3: SDS-page gel stained with Coomassie Blue.

MW: molecular weight markers (Promega)

1: Crude protein extract of strain ER2566 (Tnp)

2: Purified MBP-Tnp from strain ER2566 (Tnp). The products of Tnp breakdown appear in the form of a doublet with MW 60 kDa.

3: Crude protein extract of strain ER2566 (Tnp/pKa)

4 and 5: Two different preparations of purified MBP-Tnp from strain ER2566 (Tnp/pKa).

FIG. 4: in vitro transposition test.

Dotted line: carried out with pure MBP-Tnp produced from strain ER2566 (Tnp/pKa)

Continuous line: test carried out with pure MBP-Tnp produced from strain ER2566 (Tnp)

The experimental section below, supported by examples and figures, illustrates embodiments and advantages of the invention without being limiting in any way.

### A—MATERIALS AND METHODS

### I. Vectors

### I.1 Description

Vector pMalC2x-Tnp (Augé-Gouillou et al., 2005) derived from pMalC2x (New England Biolabs, Ozyme, Saint Quentin en Yvelines, France). This allows expression in a bacterium of a transposase fused at the N-terminal point to MPB (maltose binding protein) following induction of pLac by IPTG. The plasmid carries the ampicillin resistant gene.

Vector pET-pKa is derived from vector pET26b+ (Novagen). It allows expression in a bacterium of pKa under the control of promoter pol7. This expression is therefore restricted to bacteria which express T7 DNA polymerase such as E. coli strains BL21 or ER2566. pET-pKa carries the kanamycin resistance gene.

pBC 3Tet3 is a donor plasmid of pseudo mariner Mos1 (Augé-Gouillou et al., 2001). It contains the tetracycline resistance gene “OFF” (in other words, without a promoter) bordered by two 3′ ITRs. Transposition is detected by promoter tagging, placing the pseudo-transposon in front of the promoter, which activates resistance to tetracycline. The vector also carries the chloramphenicol resistance gene.

### I.2 Construction

1.2.1 Preparation of Vector DNA

For various constructions, all DNA elutions from agarose gel were carried out using a Wizard SV Gel and PCR Clean-Up system kit (Promega, France). All plasmid mini preparations from bacterial cultures were carried out using the Wizard Plus minipreps kit (Promega)

1.2.2 pET-pKa

The fragment coding for pKa was prepared from the CAT/pREST_{B} plasmid supplied by Dr Susan Taylor [Harward Hughes Medical Institute—USCD—La Jolla Calif. 92093—United States of America] by NdeI/HindIII digestion and eluted on 0.8% agarose gel (TAElX: 0.04M Tris-Acetate, 1 mM EDTA pH8).

The pET26b+ plasmid was directed by NdeI/HindIII, deposited on agarose gel, eluted then ligatured with the fragment coding for pKa overnight at 16° C. Control of recircularization of the plasmid onto itself was carried out by means of ligature of the plasmid in the absence of the fragment coding for pKa.

The ligation product was used to transform E. coli JM109 bacteria which were then selected on LB-kanamycin dishes (100 μg/ml). 4 ampicillin-resistant clones were cultured for plasmid extraction. DNA mini preparations were controlled by NdeI/HindIII digestion then by electrophoresis on 0.8% agarose gel (TAE 1×) in order to ensure that the plasmids were incorporated into the gene coding for pKa.

### II. Preparation of Bacterial Strains

Two ER2566 bacterial strains (New England Biolabs) were used: one (called strain Tnp) for the production of Mos1 transposase in the absence of pKa (and thus non-phosphorylated) and the other (called strain Tnp/pKa) for co-production of Mos1 transposase and pKa. In this strain, the transposase produced is phosphorylated by pKa in the bacterium.

ER2566 bacteria were transformed in order to prepare the bacterial strains:
with 100 ng of plasmid pMalC2X-Tnp (strain Tnp). Transformed bacteria were selected on agar+ampicillin (100 μg/ml).
with 100 ng of plasmid pMalC2X-Tnp+100 ng of plasmid pET-pKa strain Tnp/pKa). Transformed bacteria were selected on agar+ampicillin (100 μg/ml)+kanamycin (100 μg/ml).

### III. Production and Purification of Mos1 Transposase in ER2566 Cells (Tnp) and (Tnp/pKa)

### III.1 Production

50 ml of BBC medium (Brain Heart Infusion Broth-AES) was inoculated with 5 to 10 (Tnp) clones or 5 to 10 (Tnp/pKa) clones taken directly from the transformation dishes. The medium was supplemented with ampicillin (100 g/ml) for strain (Tnp) or ampicillin for strain (Tnp/pKa)+kanamycin (100 μg/ml) for strain (Tnp/pKa). The bacteria were immediately induced with IPTG (1 mM final) and stirred at 25° C. until a saturated culture was obtained. For strain (Tnp), culturing time was usually 16 to 20 hours while it was usually 30 to 36 hours for strain (Tnp/pKa).

### III.2 Purification

Bacterial cultures were centrifuged (5000 rpm, 10 min, 4° C.) and the residue was taken up in 5 ml of buffer (20 mM Tris, pH 9, 100 mM NaCl, 1 mM DTT). Bacteria underwent lysis by 800 μl of lysosyme at a concentration of 20 mg/ml for 30 minutes at 4° C. The bacterial lysate was centrifuged (10,000 rpm, 15 min, 4° C.) and the supernatant collected. This constituted the crude extract.

The fusion protein MBP-Tnp contained in the two types of extract (Tnp and Tnp/pKa) was purified on maltose resin in accordance with the supplier's instructions (New England Biolabs). The eluted fractions were assayed according to the Bradford method.

### IV. Analysis of Pure Mos1 Transposase Resulting from (Tnp) and (Tnp/pKa) Strains

### IV.1 Stability

The purified transposases were analysed on SDS-page gel (stacking: 4% acrylamide pH 6.8—separation: 11% acrylamide pH 8.8.). 1 to 2 μg of pure protein were deposited on the gel with a MW marker (Promega). After one hour of electrophoresis, the gels were stained with Coomassie Blue.

### IV.2 Activity

The purified transposases were tested for their ability to mediate transposition using an in vitro transposition test.

80 nM of transposase originating from either the (Tnp) line or the (Tnp/pKa) line was incubated with 600 ng of plasmid pBC3Tet3 at 30° C. in buffer (10 mM Tris, pH 9, 50 mM NaCl, 1 mM DTT, 20 mM MgCl₂, 5 mM EDTA, 10% glycerol), in the presence of 100 ng of BSA, for times ranging from 0 to 60 minutes. The reaction was stopped by 4 μg of proteinase K and 0.15% of SDS for 5 minutes at 65° C. then 30 minutes at 37° C. DNA was purified by phenol/chloroform extraction followed by precipitation in alcohol in the presence of 1 μg of tRNA. The DNA residues were taken up in 20 μl of water. 2 μl was used to transform JM109 bacteria (viable E. coli). After transformation, the culture obtained was titrated on an LB-chloramphenicol dish (150 g/ml) (40 μl of a 1/1000 dilution) and on an LB-tetracylin dish (12.5 μg/ml) (the totality (1 ml) of the culture medium was non-diluted). Dishes were incubated at 37° C. for 24 hours. The following day, colonies were counted in the LB-chloramphenicol and LB-tetracylin dishes then the frequency of transposition was calculated from the number of bacteria resistant to tetracycline compared to the total number of bacteria (cloram+tetra).

### B—RESULTS

FIG. 3 shows that the Mos1 transposase is more stable when it is produced in the presence of pKa. This stabilisation is seen by a large reduction in proteolysis. It is illustrated in FIG. 3 by the disappearance of the products of auto-proteolysis on lines 4 and 5 whereas these are highly visible on line 2 (transposase produced in the absence of pKa).

In order to verify that the transposase produced in the presence of pKa is still active in transposition, in vitro transposition tests were carried out. The results are given in FIG. 4. They show that a transposase produced in the presence of pKa (dotted line) is as effective, if not more so, than a transposase produced in the absence of pKa (continuous line). The increased efficacy of transposases produced in the presence of pKa can be directly correlated to stabilisation of the protein which does not degrade in the course of the test.

### REFERENCES

Augé-Gouillau C et al. (2001) Mol. Genet. Genomics. 265: 51-57
Lampe D J. et al. (1996) EMBO J. 15: 5470-5479
Plasterk R H A. et al. (1999) Trends in genetics 15: 326-332
Renault S. et al. (1997) Virologie 1: 133-144
Jacobson and Hartl (1985) Genetics 111: 57-65
Craig et al. (2002) Mobile DNA II. ASM Press. Washington. USA
Sambrook and Russel (2001) Molecular Cloning: a laboratory manual (3^{rd} Ed.) Cold Spring Harbor Laboratory Press, Cold Spring Harbor, N.Y.
Lohe and Hartl (1996) Mol. Biol. Evol. 13: 549-555
Jeong et al. (2002) PNAS 99: 1076-1081
Martienssen and Colot (2001) Science 293: 1070-1074
Ketting et al. (1999) Cell 99: 133-141
Tabara et al. (1999) Cell 99: 123-132
Felsenstein (1993) PHILIPS (Phylogeny Inference Package) version 3.5.c, University of Washington, Seattle
Augé-Gouillou et al. (2000) Mol. Gen. Genet. 264: 514-520
Ausubel et al. (1994) In Janssen, K. (Ed) Current Protocols in Molecular Biology. J. Wiley & Sons, INC, Massachusetts General Hospital, Harvard Medical School
Bonin et al. (2005) J. Mol. Evol. 60: 736-747
Strausberg et al. (2002) PNAS 99: 16899-16903
Augé-Gouillou et al. (2005) Mol. Cell. Biol. 25: 2861-2870

1. A process for the production by a prokaryotic host cell of an active, stable transposase of a mariner mobile genetic element belonging to the Mauritiana subfamily, comprising at least the following steps:
a) cloning of the nucleotide sequence coding for said active transposase in an expression vector;
b) cloning of a nucleotide sequence coding for the active catalytic sub-unit of cAMP-dependent protein kinase (pKa) in an expression vector;
c) transformation of said host cell with said expression vectors;
d) expression of said nucleotide sequences by said host cell; and
e) obtaining the active and stabilised transposase by pKa phosphorylation.

2. The process according to claim 1, wherein said active transposase is a hyperactive mutant transposase.

3. The process according to claim 1, wherein it also includes a purification step of the active, stable transposase obtained in step e).

4. The process according to claim 1, wherein cloning in steps a) and b) is carried out in a single expression vector.

5. The process according to claim 4, wherein the expression of said nucleotide sequence coding for the active transposase and said nucleotide sequence coding for pKa is under the control of the same regulation elements.

6. The process according to claim 1, wherein said prokaryotic host cell is a bacterial cell.

7. The process according to claim 6, wherein said bacterium is Escherichia coli.

8. The process according to claim 1, wherein said mariner mobile genetic element is Mos1.

9. An active, stable transposase of a mariner mobile genetic element belonging to the mauritiana subfamily obtainable by a process according to claim 1.

10. A prokaryotic host cell including at least:
a) the nucleotide sequence coding for an active transposase of a mariner mobile genetic element belonging to the mauritiana subfamily; and
b) the nucleotide sequence coding for the active catalytic sub-unit of cAMP-dependent protein kinase (pKa).

11. The prokaryotic host cell according to claim 10, wherein said active transposase is a hyperactive mutant transposase.

12. The prokaryotic host cell according to claim 10, wherein said nucleotide sequences a) and b) are cloned in expression vectors.

13. The prokaryotic host cell according to claim 10, wherein said nucleotide sequences a) and b) are cloned in an expression vector.

14. The prokaryotic host cell according to claim 10, wherein it is a bacterial cell.

15. The prokaryotic host cell according to claim 14, wherein said bacterium is Escherichia coli.

16. The prokaryotic host cell according to claim 10, wherein said mariner mobile genetic element is Mos1.

17. An expression vector wherein it includes at least:
a) the nucleotide sequence coding for an active transposase of a mariner mobile genetic element belonging to the mauritiana subfamily; and
b) the nucleotide sequence coding for the active catalytic sub-unit of cAMP-dependent protein kinase (pKa).

18. The expression vector according to claim 17, wherein said active transposase is a hyperactive mutant transposase.

19. The expression vector according to claim 17, wherein said mariner mobile genetic element is Mos-1.

20. A kit for insertional mutagenesis and/or sequencing and/or cloning, including at least one active, stable transposase according to claim 9.

21. A method for transposing a transposable DNA sequence of interest in a target DNA sequence, wherein at least one active, stable transposase according to claim 9 is used.

22. (canceled)
